(19) United States
(12) Patent Application Publication
(10) Pub. No.: US 2003/0012743 A1
(43) Pub. Date: Jan. 16, 2003

(54) Composition containing monoterpenes for topical oral administration

(76) Inventor: Joseph Simcha Wolnerman, Jerusalem (IL)

Correspondence Address: Joe Liebeschuetz, Townsend & Townsend & Crew, 8th Floor, Two Embarcadero Center, San Francisco, CA, 94111-3834, US

(21) Appl. No.: 10/069,416

(22) PCT Filed: Mar. 22, 2001
(86) PCT No.: PCT/IL01/00276

(30) Foreign Application Priority Data
Mar. 22, 2000 (IL) 135.220

(51) Int. Cl. A61K 35/78; A61K 9/68; A61K 7/26; A61K 31/015

(52) U.S. Cl. 424/48; 424/58; 424/736; 514/763

(57) ABSTRACT

The invention provides a composition for topical, oral administration for prevention and treatment of oral disease, comprising an active agent containing at least 70% by weight monoterpenes with three unsaturations as active agent therein, in combination with a suitable carrier.

### FIELD OF THE INVENTION

The present invention relates to a composition for topical oral administration.

More specifically, the invention relates to a composition for topical oral administration comprising monoterpenes with three unsaturations including limonene and pinene from synthetic or essential oil natural extracts in combination with a suitable carrier.

### DESCRIPTION OF THE PRIOR ART

Dental calculus, or tartar, is recognized as a recurring calcified deposit on the surfaces of teeth. It is generally recognized that dental calculus develops in a sequential process that involves the accumulation of dental plaque and the subsequent calcification of the plaque by saliva, which has very high concentrations of calcium and phosphate. Although calculus, per se, is not directly responsible for the development of oral diseases, it is recognized as a secondary, or contributing, factor in the development of periodontal disease because: (1) its presence on the teeth serves as a local irritant to the adjacent soft tissues, eliciting an inflammatory response (and soft tissue inflammation is the initial phase of periodontal disease); (2) it interferes with the normal cleansing of the tooth surfaces, which occurs during the mastication of food or through the performance of conventional oral hygiene procedures, such as toothbrushing and flossing; and (3) it harbors bacterial toxins, which exacerbate periodontal disease formation, by virtue of its porosity. Once formed, calculus deposits can only be removed through concerted mechanical procedures, i.e., a dental prophylaxis, scaling or root planing in deep pockets.

Oral tissue diseases such as gingivitis and periodontitis are a common affliction which necessitate constant care for prevention and treatment. The domestic personal use of a toothbrush, toothpaste, mouthwash, dental floss and dental tooth picks are recommended for removing food particles, cleaning tooth surfaces and stimulating the gums.

While a toothbrush has a primary function of removing food particles, the toothpaste has secondary function. The toothpaste is provided to encourage brushing by its texture, flavor and odor.

The texture dampens the rigid-dry sensation of the toothbrush, while the flavoring and fragrant components mask the taste of therapeutic substances within the toothpaste. An example of such a substance Is a detergent aimed at assisting the removal of fats which adhere to the teeth.

The prior art discloses compositions and methods that use oxidizing agents, antimicrobial agents, and antibiotics for the treatment of various oral care conditions. Most of these prior art references teach that the delivery of these agents is essential to provide efficacy. This is in contrast to the present invention which focuses on the delivery of natural agents, monoterpenes with three unsaturations, that unexpectedly show a strong activity to the oral cavity, to provide efficacy.

The prior art teaches a variety of ways to deliver oxidation agents, in oral care compositions, to the oral cavity. For example, U.S. Pat. Nos. 4,689,215 issued Aug. 25, 1987; 4,837,009 issued Jun. 6, 1989; 4,696,811, issued Sep. 29, 1987; 4,808,389 issued Feb. 28, 1989; 4,786,492 issued Nov. 22, 1988; 4,788,053 issued Nov. 29, 1988; 4,792,442 issued Dec. 20, 1988; 4,818,519 issued Apr. 4, 1989; 4,851,21 issued Jul. 25, 1989; 4,855,135 issued Aug. 8, 1989; 4,793,989 issued Dec. 27, 1988; 4,886,657 issued Dec. 12, 1989; 4,889,714 issued Dec. 26, 1989; 4,925,656 issued May 15, 1990; 4,975,285 issued Dec. 4, 1990; 4,978,535 issued Dec. 18, 1990; 5,200,171 issued Apr. 6, 1993; 5,348,734 issued Sep. 20, 1994; 5,618,550 issued Apr. 8, 1997, and 5,489,435 issued Feb. 6, 1996, all to Perry A. Ratcliffe, teach oral care compositions and methods of treatment using stabilized chlorine dioxide.

Compositions containing limonene among a list of components have been mentioned in the prior art. For example, U.S. Pat. No. 5,453,276 lists limonene among anacerdic acid, farnesol, citronellol, pine resin, hinokitiol, longifolene and caryophyllene that together showed antimicrobial activity.

U.S. Pat. Nos. 5,279,813, 5,273,741, 5,234,688, 5,167,951, describes antiplaque compositions containing triclosan as antibacterial agent and polyphosphate for anti-tartar actions. In these compositions, limonene was used as stabilizer. U.S. Pat. No. 5,910,455 mentions limonene as an abrasive hand cleansing material in a cleanser compostion. Limonene is also mentioned in U.S. Pat. No. 5,079,063 as antiflea agent for making fliea-free carpets. Limonene is also described in U.S. Pat. No. 5,164,416 as skin penetrating enhancer in transdermal formulations. All of the above references are incorporated herein by reference in their entirety.

U.S. Pat. No. 5,939,050 discloses a combination of two groups of materials that produce a synergistic antimicrobial effect. The compounds include natural and synthetic copounds such as berberine, cedarwood oil, chloramphenicol, citral, citronella oil, cocamidopropyl, dimethylglycine, and lemon oil. The lemon oil shows low antimicrobial activity but in combination with the other compounds it shows a synergistic antimicrobial effect. The lemon oil is used in a very low concentration in the final formulation of said patent. Said patent does not teach or suggest the present discovery that, inter alia, lemon oil has an unexpected activity for improving gingivitis which is not necessarily related to the antimicrobial activity which has been shown to be very low for lemon oil when lemon oil was used alone in said patent. Thus, the focus of said patent is antimicrobial activity focussing on certain components working in combination and, as indicated, said patent does not teach or suggest to a person skilled in the art that any advantage is achieved by using lemon oil or an extract thereof by itself as the primary active ingredient.

The above prior art references have not recognized that the delivery of monoterpenes with three unsaturations to the oral cavity will provide efficacy in various oral care conditions. Because prior art references have focused on the delivery of chlorine dioxide for efficacy, prior art compositions and methods of treatment may have various drawbacks.

Therefore, prior art compositions, mentioned above, have not been entirely satisfactory for the treatment and/or prevention of gingivitis, plaque, and periodontal disease. Therefore, additional efficacious compositions and methods of treatment for these purposes are desirable.

As mentioned above, the present invention relates to the delivery of monoterpenes with three unsaturations to the oral cavity for efficacy. It is the purpose of the present invention to provide compositions and methods for treating or preventing diseases of the oral cavity, such as plaque, gingivitis, and periodontal disease, by utilizing an effective amount of monoterpenes of three unsaturations wherein present in the oral care composition at the time of use.

Further, the present invention relates to oral care compositions, including therapeutic rinses, especially mouth rinses, as well as toothpastes, tooth gels, tooth powders, non-abrasive gels, chewing gums, mouth sprays, and lozenges (including breath mints). These compositions comprise a minimally effective amount of Limonene.

These compositions are effective in killing, and/or altering the bacterial metabolism, and/or for a period of time suppressing the growth of, microorganisms which cause topically-treatable infections and diseases of the oral cavity, such as plaque, gingivitis, periodontal disease, and breath malodor. These compositions are also effective to affect inflammation.

### SUMMARY OF INVENTION

According to the present invention there is now provided a composition for oral cavity treatment comprising monoterpenes with three unsaturations from natural or synthetic source as active agents in combination with a suitable carrier for prevention and treatment of oral cavity diseases.

More specifically, the present invention now provides a composition for topical, oral administration for prevention and treatment of oral disease, comprising an active agent containing at least 70% by weight monoterpenes with three unsaturations as active agent therein, in combination with a suitable carrier.

In a preferred embodiment of the present invention the composition of the active agent comprises at least 70% by weight of monoterpenes of three unsaturations with at least 60% is limonene.

In an even further preferred embodiment of the present invention monoterpenes are from natural source.

In a most preferred embodiment the monoterpenes are of essential oils of citrus.

In preferred embodiments of the present invention said essential oils is selected from the group consisting of lemon, pomella and citron comprising 70% by weight monoterpenes with three unsaturations or more.

In a most preferred embodiment of the present invention said essential oil of a citron.

In another aspect of the invention, a synergistic effect is obtained by combining limonene and monoterpenes with Carnallite or salts thereof in a synergistic and effective amount.

Thus, in another preferred embodiment of the present invention said extract is combined with a salt selected from the group consisting of MgCl₂, MgBr₂, NaCl, KCl, CaCl₂ and mixtures thereof.

In a preferred embodiment of the invention the Carnallite is present in an amount of about 5-99% wt/wt.

In a further preferred embodiment the composition has a saline concentration of about 50% and an effective amount of said extract is added immediately before application.

The composition of the present invention will be combined with a suitable carrier selected from the group consisting of toothpaste, mouthwash, lozenges, chewing gum and toothpowder.

In a preferred embodiment of the present invention monoterpenes of three unsaturations is present in an amount of up to 10% wt/wt.

In another aspect of the invention there is provided a method for oral treatment, consisting of administering to a patient a composition comprising monoterpenes of three unsaturations in combination with a suitable carrier.

In a preferred embodiment there is provided a method for oral treatment, consisting of administering to a patient a composition comprising monoterpenes of three unsaturations further comprising Carnallite in a synergistic amount, in combination with a suitable carrier.

In another aspect of the invention there is provided a method for oral treatment, consisting of administering to a patient a composition comprising an essential oil of a citron fruit in combination with a suitable carrier.

In a preferred embodiment there is provided a method for oral treatment, consisting of administering to a patient a composition comprising an essential oil of a citron fruit further comprising Carnallite in a synergistic amount, in combination with a suitable carrier.

The present invention further provides a method for treating and/or preventing plaque, dental calculus, gingivitis, periondontitis, and oral viral diseases.

In an even further preferred embodiment there is provided a method for reducing the depth of periodontal pockets in a patient, comprising administering any of the compositions mentioned above.

The extraction of the essential oil of the present invention was achieved utilizing steam distillation. In this process fresh peels were utilized resulting in about 1.5% by weight of essential oil in comparison to the original of said peels.

The monoterpene and essential oil active agents of the present invention, preferably in combination with a Carnallite salt, can also be microencapsulated by methods and with components known per se.

As stated hereinbefore, the present invention relates to compositions and methods of treating or preventing diseases of the oral cavity (e.g. plaque, gingivitis, periodontal disease), in humans or animals, by applying to the oral cavity, a safe and effective amount of monoterpenes of three unsaturations.

The term “monoterpenes of three unsaturations” as used herein, is meant to denote a composition containing at least one monoterpene of three unsaturations of the molecular formula C₁₀H₁₆, wherein unsaturation refers to a double bond or a cyclization. Examples are limonene containing two double bonds and one cyclic group, myrcene containing three double bonds, and sabinene, a-pinene and b-pinene.

“Safe and effective amount” as used herein is meant to denote an amount of monoterpene with three unsaturation derivatives, high enough to significantly (positively) modify the condition to be treated, but low enough to avoid serious side effects (at a reasonable benefit/risk ratio), within the scope of sound medical/dental judgment. The safe and effective amount of monoterpenes, will vary with the particular condition.

“Toothpaste” as used herein is meant to denote paste, powder, and tooth gel formulations unless otherwise specified.

“Oral care composition” or “oral composition” as used herein is meant to denote a product which is not intentionally swallowed for purposes of systemic administration of therapeutic agents, but is retained in the oral cavity for a sufficient time to contact substantially all of the dental surfaces and/or oral mucosal tissues for purposes of oral activity.

A “pharmaceutically-acceptable excipient” or “pharmaceutically-acceptable oral carrier,” as used herein, is meant to denote one or more compatible solid or liquid filler diluents or encapsulating substances which are suitable for topical, oral administration.

“Compatible,” as used herein, is meant to denote that the components of the composition are capable of being commingled without interaction in a manner which would substantially reduce the composition's stability and/or efficacy for treating or preventing breath malodor, plaque, gingivitis, and periodontal disease, according to the compositions and methods of the present invention.

The carriers or excipients of the present invention can include the usual and conventional components of toothpastes (including gels and gels for subgingival application), mouth rinses, mouth sprays, chewing gums, and lozenges (including breath mints) as more fully described hereinafter. The compositions of the present invention can be dual phase compositions or single phase compositions.

The choice of a carrier to be used is basically determined by the way the composition is to be introduced into the oral cavity. If a toothpaste (including tooth gels, etc.) is to be used, then a “toothpaste carrier” is chosen as disclosed in, e.g., U.S. Pat. No. 3,988,433, to Benedict, the disclosure of which is incorporated herein by reference (e.g., abrasive materials, sudsing agents, binders, humectants, flavoring and sweetening agents, etc.). If a mouth rinse is to be used, then a “mouth rinse carrier” is chosen, as disclosed in, e.g., U.S. Pat. No. 3,988,433 to Benedict (e.g., water, flavoring and sweetening agents, etc.). Similarly, if a mouth spray is to be used, then a “mouth spray carrier” is chosen or if a lozenge is to be used, then a “lozenge carrier” is chosen (e.g., a candy base), candy bases being disclosed in, e.g., U.S. Pat. No. 4,083,955, to Grabenstetter et al., which is incorporated herein by reference; if a chewing gum is to be used, then a “chewing gum carrier” is chosen, as disclosed in, e.g, U.S. Pat. No. 4,083,955, to Grabenstetter et al., which is incorporated herein by reference (e.g., gum base, flavoring and sweetening agents). Carriers suitable for the preparation of compositions of the present invention are well known in the art. Their selection will depend on secondary considerations like taste, cost, and shelf stability, etc.

Preferred compositions of the subject invention are in the form of toothpastes and tooth gels. Components of such toothpaste and tooth gels generally include one or more of a dental abrasive (from about 10% to about 50%), a surfactant (from about 0.5% to about 10%), a thickening agent (from about 0.1% to about 5%), a humectant (from about 10% to about 55%), a flavoring agent (from about 0.04% to about 2%), a sweetening agent (from about 0.1% to about 3%), a coloring agent (from about 0.01% to about 0.5%) and water (from about 2% to about 45%). Such toothpaste or tooth gel may also include one or more of an anticaries agent (from about 0.05% to about 0.3% as fluoride ion), and an anticalculus agent (from about 0.1% to about 13%).

Other preferred compositions of the subject invention are mouthwashes, including mouth sprays. Components of such mouthwashes and mouth sprays typically include one or more of water (from about 45% to about 95%), ethanol (from about 0% to about 25%), a humectant (from about 0% to about 50%), a surfactant (from about 0.01% to about 7%), a flavoring agent (from about 0.04% to about 2%), a sweetening agent (from about 0.1% to about 3%), and a coloring agent (from about 0 001% to about 0.5%). Such mouthwashes and mouth sprays may also include one or more of an anticaries agent (from about 0.05% to about 0.3% as fluoride ion), and an anticalculus agent (from about 0.1% to about 3%).

Other preferred compositions of the subject invention are dental solutions. Components of such dental solutions generally include one or more of water (from about 90% to about 99%), preservative (from about 0.01% to about 0.5%), thickening agent (from 0% to about 5%), flavoring agent (from about 0.04% to about 2%), sweetening agent (from about 0.1% to about 3%), and surfactant (from 0% to about 5%).

The compositions of the present invention are preferably essentially free of organic solvents. The compositions of the present invention are also preferably essentially free of peroxy compounds.

Types of carriers or oral care excipients which may be included in compositions of the present invention, along with specific non-limiting examples, are:

Abrasives:

Dental abrasives useful in the topical, oral carriers of the compositions of the subject invention include many different materials. The material selected must be one which is compatible within the composition of interest and does not excessively abrade dentin. Suitable abrasives include, for example, silicas including gels and precipitates, insoluble sodium polymetaphosphate, hydrated alumina, calcium carbonate, dicalcium orthophosphate dihydrate, calcium pyrophosphate, tricalclum phosphate, calcium polymetaphosphate, and resinous abrasive materials such as particulate condensation products of urea and formaldehyde.

Another class of abrasives for use in the present compositions is the particulate thermo-setting polymerized resins as described in U.S. Pat. No. 3,070,510 issued to Cooley & Grabenstetter on Dec. 25, 1962. Suitable resins include, for example, melamines, phenolics, ureas, melamine-ureas, melamine-formaldehydes, urea-formaldehyde, melamine-urea-formaldehydes, cross-linked epoxides, and cross-linked polyesters. Mixtures of abrasives may also be used.

Silica dental abrasives of various types are preferred because of their unique benefits of exceptional dental cleaning and polishing performance without unduly abrading tooth enamel or dentine. A particularly preferred precipitated silica is the silica disclosed in U.S. Pat. Nos. 5,603,920, issued on Feb. 18, 1997; 5,589,160, issued Dec. 31, 1996; 5,658,553, issued Aug. 19, 1997; 5,651,958, issued Jul. 29, 1997, all of which are assigned to the Procter & Gamble Co. All of these patents are incorporated herein by reference in their entirety.

Mixtures of abrasives can be used. All of the above patents regarding dental abrasives are incorporated herein by reference. The total amount of abrasive in dentifrice compositions of the subject invention preferably range from about 6% to about 70% by weight; toothpastes preferably contain from about 10% to about 50% of abrasives, by weight of the composition. Solution, mouth spray, mouthwash and non-abrasive gel compositions of the subject invention typically contain no abrasive.

Suitable surface active agents are those which are reasonably stable and form foam throughout a wide pH range. Surface active agents include nonionic, anionic, amphoteric, cationic, zwitterionic, synthetic detergents, and mixtures thereof. Many suitable nonionic and amphoteric surfactants are disclosed by U.S. Pat. Nos. 3,988,433 to Benedict; U.S. Pat. No. 4,051,234, issued Sep. 27, 1977, and many suitable nonionic surfactants are disclosed by Agricola et al., U.S. Pat. No. 3,959,458, issued May 25, 1976, both incorporated herein in their entirety by reference.

Nonionic and Amphoteric Surfactants

Nonionic surfactants which can be used in the compositions of the present invention can be broadly defined as compounds produced by the condensation of alkylene oxide groups (hydrophilic in nature) with an organic hydrophobic compound which may be aliphatic or alkyl-aromatic in nature. Examples of suitable nonionic surfactants include poloxamers (sold under trade name Pluronic), polyoxyethylene sorbitan esters (sold under trade name Tweens), fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, and mixtures of such materials.

The amphoteric surfactants useful in the present invention can be broadly described as derivatives of aliphatic secondary and tertiary amines in which the aliphatic radical can be a straight chain or branched and wherein one of the aliphatic substituents contains from about 8 to about 18 carbon atoms and one contains an anionic water-solubilizing group, e.g., carboxylate, sulfonate, sulfate, phosphate, or phosphonate. Other suitable amphoteric surfactants are betaines, specifically cocamidopropyl betaine. Mixtures of amphoteric surfactants can also be employed. The present composition can typically comprise a nonionic, amphoteric, or combination of nonionic and amphoteric surfactant each at a level of from about 0.025% to about 5%.

Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed. The present composition typically comprises an anionic surfactant at a level of from about 0.025% to about 9%, preferably from about 0.05% to about 7%, and most preferably from about 0.1% to about 5%.

Flavoring agents can also be added to the compositions. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, propenyl guaethol, cinnamon, vanillin, thymol, linalool, cinnamaldehyde glycerol acetal known as CGA, and mixtures thereof. Flavoring agents are generally used in the compositions at levels of from about 0.001% to about 5%, by weight of the composition.

Sweetening agents which can be used include sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame and cyclamate salts, especially sodium cyclamate and sodium saccharin, and mixtures thereof. A composition preferably contains from about 0.1% to about 10% of these agents, preferably from about 0.1% to about 1%, by weight of the composition.

In addition to flavoring and sweetening agents, coolants, salivating agents, warming agents, and numbing agents can be used as optional ingredients in compositions of the present invention. These agents are present in the compositions at a level of from about 0.001% to about 10%, preferably from about 0.1% to about 1%, by weight of the composition. The coolant can be any of a wide variety of materials. Included among such materials are carboxamides, menthol, ketals, diols, and mixtures thereof.

Preferred salivating agents of the present invention include Jambus.RTM. manufactured by Takasago. Preferred warming agents include capsicum and nicotinate esters, such as benzyl nicotinate. Preferred numbing agents include benzocaine, lidocaine, clove bud oil, and ethanol.

It is preferred that the mouth rinse to be taken into the oral cavity have a concentration of monoterpenes of three unsaturations in the range of from about 0.04% to about 5.0%, with from about 0.1% to about 2.0%, by weight of the composition, being even more preferred.

Mouth sprays preferably may have monoterpenes of three unsaturations from about 0.15% to about 10%, with from about 0.2% to about 5% more preferred.

Preferably for dentifrices (including toothpaste and tooth gels) and non-abrasive gels, the concentration of monoterpenes of three unsaturations is in the range of from about 0.4% to about 4.5%, by weight of the composition, with from about 0.75% to about 3% preferred, and from about 1.5% to about 2%, by weight of the composition, being even more preferred.

For the method of treating diseases or conditions of the oral cavity of the present invention, a safe and effective amount of monoterpenes of three unsaturations is preferably applied to the gingival/mucosal tissue and/or the teeth (for example, by rinsing with a mouthrinse, directly applying a non-abrasive gel with or without a device, applying a dentifrice or a tooth gel with a toothbrush, sucking or chewing a lozenge or breathmint, etc.) preferably for at least about 10 seconds, preferably from about 20 seconds to about 10 minutes, more preferably from about 30 seconds to about 60 seconds. The method often involves expectoration of most of the composition following such contact. The frequency of such contact is preferably from about once per week to about four times per day, more preferably from about thrice per week to about three times per day, even more preferably from about once per day to about twice per day. The period of such treatment typically ranges from about one day to a lifetime. For particular oral care diseases or conditions the duration of treatment depends on the severity of the oral disease or condition being treated, the particular delivery form utilized and the patient's response to treatment. If delivery to the periodontal pockets is desirable, such as with the treatment of periodontal disease, a mouth rinse can be delivered to the periodontal pocket using a syringe or water injection device. These devices are known to one skilled in the art.

Active Ingredient A

Essential oils from citrus, particularly of Citron Fruit composed of at least 65% monoterpenes with three unsaturations.

Active Ingredient B

CARNALLITE having the following characteristics:

| Minerals | Grams/liter |
|---|---|
| MgCl₂ | 170-240 |
| NaCl | 15-25 |
| KCl | 13-24 |
| CaCl₂ | 40-58 |
| MgBr₂ | 5-12 |

The Carnallite is effective in fluid absorption and in the reduction of swelling and edema. These effects are facilitated by the changes in osmotic pressure affected by the presence of the above minerals. The magnesium and the bromide have antiseptic and anti-inflammatory properties.

Silica can also be added to the above list. The silica portion of the formulation functions as a soft abrasive and polishing agent. It also has mechanical functions such as removal of dental plaque, bacteria and food particles and tends to brighten the enamel.

The potassium and calcium, in combination with fluoride (not mentioned above), induce remineralization of the bone and of the dentine which was affected by caries at the cervical area of the tooth surface. As a result, there is a decrease in the cervical sensitivity of the teeth.

The fluid absorption facilitated by salt concentration has a stimulating effect on the gingival fluid thereby creating an increased secretion/production of immunal globulins. As a result, the immunal globulin concentration within the gingivital fluid is much higher than the concentration found within the saliva. This phenomenon prevents the development of anaerobic bacteria found within the gingival pocket.

The natural materials of the present invention serve as stimulators for the immune system. They also have a local effect; they encourage the secretion of gingival fluid to gingival pockets.

Like with other homeopathic medicines, using the compositions of the present invention raises the level of immunoglobolins in the saliva, with a rise in the general saliva secretion. This prevents the pathogenic bacteria from sticking to the teeth and detains their growth and proliferation.

The above compositions encourage the creation of a new periodontal ligament replacing the ligament which has been destroyed during the Periodontitis disease by widening blood vessels, proliferating capillary blood vessels, enlarging the perfusion and remineralizing the bone and the periodontal cement.

The present invention may eliminate the use of various detergents which are a common component of toothpaste. This may resolve the problem of the unspecified inflammatory reaction that often afflicts those using regular toothpaste. One can find in the dental literature a description of the linkage between Aphtos Stomatitus and a decline in the immune reaction of the mouth and the prevalence of detergents in toothpaste.

While the invention will now be described in connection with certain preferred embodiments in the following examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

### EXAMPLE 1

### Biological Effect of Limonene, Pinene and Myrcene Oil on Cells

The tests concentrated on the active agent (i.e drug) effects on anaerobic bacteria that are prevalent in oral cavity and which are known to cause damage. Two bacteria were selected: a. Porphyromonas gingivalis and b. Fusobacterium nucleatum.

Murine monocytes, J774 were used to determine possible effects on eukaryotic tissue. Leishmania major promastigotes served as a parasite and a non-specific eukariotic control. Drug effects which might be related to anti- or pro-inflammatory processes were conducted. The effects on IL-10, IL-12, Interferon χ (IFN-χ) and tumor necrosis factor (TNF) were examined.

Tests were made to determine the optimal amounts of cells or bacteria needed for estimation of the drug effects. The drugs that were used comprised limonene, about 90% by weight, and about 2% of a mixture of myrcene, a and b pinenes. The examined compounds, were highly effective in all trials.

Effect on J774 Murine Monocytes

7500 cells were seeded in 200 μl wells. 24 hours later the compounds were added. The drug and control (saline) were diluted 1/1 in ethanol and further diluted in ethanol. 2 μl of the dilutions were added to the cultures. Carnallite concentration was 2.5 mg/ml. Cells and drugs were incubated for 48 hours together.

Drug effect was estimated by incorporation of tritiated thymidine which was added (0.5μ Ci/25 μl/each well) 18 hours before harvesting the culture in a cell harvester and measuring the radioactivity with a scintillation counter. The ED 50 of the drug is about 1/40,000. The ethanol solvent and saline control showed no effect.

Effect on Leishmania major Promastigotes

150,000 promastigotes and drugs were incubated in 200 μl medium for 48 hours. The drugs were added in 2 ? l and Thymidine was added as previously described. The culture was harvested after 48 hours.

Two citrus essential oil compositions were tested, one containing 92% limonene and the second 75% limonene.

The 92% limonene compound had an ED 50 of about 1/50,000 dilution and the 75% had an ED 50 of about 1/35,000 dilution. When Carnallite was added to the limonene compositions, an increase of about 10% of the activity was recognized.

Effect on Oral Bacteria.

Medium suitable for anaerobic bacterial growth was kept in anaerobic conditions for 24 hours after which bacteria and drugs were added bacterial growth was estimated 24 hours later by spectroscopy.

Two series of experiments were conducted with Fusobacterium nucleatum.

a. In the first series we examined natural limonene as part of Citron essential oil (92% limonene) and mixture of Citron essential oil and Carnallite. The Limonene from Citron oil at a dilution of 1/400 inhibited about 50% of bacterial growth, the Carnallite slightly increased the activity of the oil and inhibited 60% of bacterial growth under similar concentration and conditions.

One experiment was conducted with Porphyromonas gingivalis.

The choice of drug dilutions was based on the results with Fusobacterium nucleatum. Dilution of 1/1200 killed all the bacteria.

Effects on Cytokine Production

200,000 mononuclear cells were incubated with Citron oil containing 92% limonene for 18 hours after which cytokines released to the supernatant were determined. All determinations were performed by Elisa using specific anticytokine monoclonal antibodies. There was no spontaneous IFN χ production in control or treated cells.

The Limonene compound which was diluted 1/300 increased IL-10, and decreased IL-12 and TNF release. This means that some anti-inflammatory processes were blocked.

Conclusions

The Limonene compositions had an effect of ED 50 when diluted 1/20000-1/50000, against J774 mouse monocytic cell line and promastigotes of Leishmania major Concentrations of 1/400-1/1200 killed at least 50% of anaerobic bacteria.

TABLE 1
Summarizes the effects on L. major promastigotes

| | 1/45000 | 1/15000 | 1/5000 |
|---|---|---|---|
| Carnallite | — | — | — |
| Limonene, | 5 | 56 | 81 |
| 92% | | | |
| Limonene, | 16 | 68 | 82 |
| 92% + Carnal | | | |
| saline | 14 | 7 | 14 |
| Saline + | 26 | 22 | 34 |
| Carnallite | | | |

The results are percent inhibition of thymidine incorporation.
**Limonene composition was diluted in ethanol. Ethanol had no inhibitory activity.

### EXAMPLE 2

### Formulations

Various liquid formulations were prepared using monoterpene oil as main active agent:

The following examples are made by conventional processes by mixing the following:

Example 2a--Single Phase Dentifrice

| Ingredient | Wt. % |
|---|---|
| Water | 65.00 |
| Limonene oil | 4.000 |
| Sodium Fluoride | 0.200 |
| Hydrated Silica | 25.00 |
| Xanthan Gum | 0.600 |
| Sodium dodecyl sulfate | 4.000 |
| (27.9% Sol'n) | |
| Titanium Dioxide | 0.500 |
| Sodium Saccharin | 0.200 |
| Flavor | 0.500 |
| Total | 100.00 |

Example 2b--Mouthwash

| Ingredient | Wt % |
|---|---|
| Water | 98.00 |
| Limonene oil (75%) | 1.25 |
| Tween 20 | 0.50 |
| Flavor | 0.25 |
| Total | 100.00 |

Example 2c--Limonene Lozenge
Ingredient

| | | |
|---|---|---|
| Limonene oil | 6 | mg. Per lozenge |
| Flavor | As desired | |
| Magnesium Stearate | 7.5 | mg. |
| Stearic Acid | 75 | mg. |
| Compressible Sugar | QS 1500 | mg. |

Example 2d
Non-Abrasive Gel

| Ingredient | Weight % |
|---|---|
| Limonene (80%) | 6.00 |
| Tween 80 | 0.25 |
| Carboxymethyl cellulose | 10.00 |
| Crystalline cellulose | 1.00 |
| Sodium Bicarbonate | 0.75 |
| Water | QS 100% |

Disperse the Carboxymethyl cellulose in water. Thereafter, add the sodium bicarbonate and mix. Then add the Tween along with the limonene oil and mix

### EXAMPLE 3

A 6-month trial was carried out, utilizing the three following components and combinations thereof:

| | |
|---|---|
| Component A: | An extract of a citron fruit |
| Component B: | Carnallite |
| Component C: | |

Plant extracts from:

(1) Salvia (fruticosa)

(2) Junjerus

Component A was prepared as follows: The citron leaves and the citron outer peel were boiled in water (100° C.) for up to one minute, crushed in blender and filtered. The resulting fluid was added to a salt solution having a final salt concentration of 25%.

The periodontal improvement was measured according to the Periodontal screening Index (PSI) and the results are presented in Table 1:

TABLE 1

| | | | | | | Point of |
|---|---|---|---|---|---|---|
| Component | A + B | B + C | C | A | B | Origin |
| PSI | 3.04 | 1.33 | 1.15 | 1.25 | 1.21 | 1 |

As can be seen, the citron extract produced better results than the Carnallite (Component B), or Component C alone, however when the citron extract (A) was combined with the Carnallite (B) the results were about three-fold greater than any of the individual components.

### EXAMPLE 4

Maximum effectiveness is accomplished in a salt solution having a concentration of 50% in an aqueous solution when adding the active ingredient A a short period before brushing. The preferred application would include the addition of one drop of ether oil onto the toothbrush shortly before brushing.

Although the effectiveness of said solution is practically instantaneous, the long-term therapeutic effect on the gums is apparent after two weeks, while the periodontal screening index (PSI), continues to increase (indicating an improvement) throughout the duration of applying the above composition

The first observable phenomenon upon initiating treatment with the above solution is the disappearance of bleeding from the gums. In addition, during a probing procedure (the measurement of pocket depth), the bleeding effects are significantly lower. At a later stage, the teeth appear to be more rigidly anchored within the gums.

As will be seen in Table 2 below, an impressive result is the reduction in pocket depth. This result is apparent within the first week of treatment and achieves peak results after a month.

TABLE 2

| Pocket | | Reduced | | | | | | |
|---|---|---|---|---|---|---|---|---|
| Reduction | | Bleeding | | Gingival Index | | Plaque Index | | |
| A + B | Control | A + B | Control | A + B | Control | A + B | Control | Week |
| 84 | 92 | 6 | 8 | 1.05 | 0.95 | 1.35 | 2.9 | 1 |
| 75 | 92 | 2 | 9 | 1.1 | 0.75 | 0.8 | 2.85 | 2 |
| 62 | 96 | 0 | 10 | 1.05 | 0.78 | 0.8 | 2.9 | 4 |
| 56 | 92 | 0 | 10 | 1.15 | 0.8 | 1.0 | 2.8 | 12 |

| | | | | |
|---|---|---|---|---|
| 100 | 10 | 1.2 | 3.2 | Baseline |

The overall results point to the following attributes:

1. The formula prevents the creation of plaque and dental calculus.

2. The formula cures gum diseases. Gingivitis as well as Periodontitis. Bleeding from the gums stops soon after initiating treatment. The depth of the periodontal pockets is reduced to half within one to two weeks of treatment.

3. The formula quickens the healing processes of inflammatory phenomenon of the oral cavity, in situations of viral diseases such as Acute Necrotizing Ulcerative Gingivitis and Recurrent Aphtosis.

4. The materials have proven to be safe and efficient.

5. In certain situations, we observed the phenomenon of remineralization of the tooth in areas of caries, especially in the cervical area of the tooth.

6. With the curing of the inflammations, the situation of those suffering from an unpleasant odor from the oral cavity (Fetor-Ex-Ore) had improved immensely.

### EXAMPLE 5

Using solutions having a concentration of 10% the salts presented in table 3 below were applied after tooth brushing, in the form of a mouth wash. The mouth wash was applied for 30 seconds, twice a day, for a week. The following results were measured according to the periodontal index (PI):

TABLE 3

| | | | | | Point of |
|---|---|---|---|---|---|
| Salts | Carnallite | Mg Br₂ | KcI | NaCl | Origin |
| PI | 1.87 | 1.58 | 1.47 | 1.32 | 1 |

### EXAMPLE 6

Aromatic oils of each of the fruits represented within Table 4 below were added to carnallite salt solutions having a salt concentration of 25%. The solutions were used for brushing (the teeth) for one minute, twice a day during two weeks. The following results were measured according to the periodontal index (PI):

TABLE 4

| Component | | | | | |
|---|---|---|---|---|---|
| B in addition | | | | | Point of |
| to | Orange | Pomella | Lemon | Citron | Origin |
| | 1.15 | 1.77 | 2.02 | 2.98 | 1 |

It will be evident to those skilled in the art that the invention is not limited to the details of the foregoing illustrative embodiments and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed is:

1. A composition for topical, oral administration for prevention and treatment of oral disease, comprising an active agent containing at least 70% by weight monoterpenes with three unsaturations as active agent therein, in combination with a suitable carrier.

2. A composition according to claim 1, wherein said active agent is a natural or synthetic mixture consisting of limonene, myrcene, a-pinene, b-pinene, sabinene and mixtures thereof, of which at least 60% by weight is limonene.

3. A composition according to claim 1, wherein said monoterpenes with three unsaturations is extracted from a citrus fruit selected from the group consisting of lemon, pomella and citron.

4. A composition according to claim 1 for topical oral administration for prevention and treatment of oral disease comprising an extract of a citrus fruit containing at least 60% Limonene in combination with a suitable carrier.

5. A composition according to claim 4, wherein said extract is an extract of the outer peel of said fruit.

6. A composition according to claim 5, wherein said extract is an aromatic oil.

7. A composition according to claim 6, wherein said extract is an ether oil.

8. A composition according to claim 4, wherein said fruit is selected from the group consisting of lemon, pomella and citron.

9. A composition according to claim 4, wherein said fruit is a citron.

10. A composition according to claim 1, further comprising a salt selected from the group consisting of MgBr₂, MgCl₂, NaCl, KCl, CaCl₂ and mixtures thereof.

11. A composition according to claim 1, further comprising Carnallite in a synergistic and effective amount.

12. A composition according to claim 11, wherein said Carnallite is present in an amount of about 5-99% wt/wt of the active composition.

13. A composition according to claim 1, wherein said suitable carrier is selected from the group consisting of toothpaste, mouthwash, lozenges, chewing gum and toothpowder.

14. A composition according to claim 1, wherein said monoterpenes are present in the carrier in an amount of up to 10% wt/wt.

15. A composition according to claim 1, wherein said monoterpenes are present in an amount of up to 2% wt/wt

16. A method for oral treatment, consisting of administering to a patient a composition comprising a terpenoid oil consisting of at least 65% limonene oil in combination with a suitable carrier.

17. A method for treating and/or preventing plaque in a patient, comprising administering a composition of claim 5.

18. A method for treating and/or preventing dental calculus in a patient, comprising administering a composition of claim 5.

19. A method for treating and/or preventing gingivitis in a patient, comprising administering a composition of claim 5.

20. A method for treating and/or preventing periodontitis in a patient, comprising administering a composition of claim 5.

21. A method for reducing the depth of periodontal pockets in a patient, comprising administering a composition of claim 5.

22. A method according to claim 5, wherein said suitable carrier is selected from the group consisting of toothpaste, mouthwash, lozenges, chewing gum and tooth powder.

23. A mouth rinse having a concentration of monoterpenes of three unsaturations in the range of from about 0.04% to about 5.0%, by weight of the composition.

24. A mouth rinse having a concentration of monoterpenes of three unsaturations in the range of from about 0.1% to about 2.0%, by weight of the composition.

25. Mouth sprays having a concentration of monoterpenes of three unsaturations from about 0.15% to about 10%, by weight of the composition.

26. Mouth sprays having a concentration of monoterpenes of three unsaturations with from about 0.2% to about 5%, by weight of the composition.

27. Toothpaste and gels having a concentration of monoterpenes of three unsaturations in the range of from about 0.4% to about 5.0%, by weight of the composition.

28. Toothpaste and gels having a concentration of monoterpenes of three unsaturations in the range of from about 0.75% to about 3% by weight of the composition.

29. Toothpaste and gels having a concentration of monoterpenes of three unsaturations in the range of from about 1.5% to about 2%, by weight of the composition.
